SEQUENCE LISTING (1 sequence)

SEQ ID NO: 1 - protein, 18 aa, Artificial Sequence
  Linker sequence
SGSSLTGTRSDIGPSRAT

(19) United States
(12) Patent Application Publication
(10) Pub. No.: US 2009/0075309 A1
(43) Pub. Date: Mar. 19, 2009

(54) Bisdeoxycoelenterazine derivatives, methods of use, and BRET2 systems

(76) Inventors: Sanjiv S. Gambhir, Portola Valley, CA (US); Jelena Levi, Palo Alto, CA (US); Abhijit De, Mountain View, CA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/283,092

(22) Filed: Sep. 8, 2008

Related U.S. Application Data
(60) Provisional application No. 60/970,280, filed on Sep. 6, 2007.

(51) Int. Cl. C12Q 1/66; C07D 487/04

(52) U.S. Cl. 435/8; 544/349

(57) ABSTRACT

Embodiments of the present disclosure provide for: compositions, BRET systems, kits, and the like.

### CROSS-REFERENCE TO RELATED APPLICATION

This application claims priority to U.S. provisional application entitled, “BISDEOXYCOELENTERAZINE DERIVATIVES, METHODS OF USE, AND BRET2 SYSTEMS,” having Ser. No. 60/970,280, filed on Sep. 6, 2007, which is entirely incorporated herein by reference.

### STATEMENT REGARDING FEDERALLY SPONSORED RESEARCH OR DEVELOPMENT

This invention was made with government support under Grant No.: NCI ICMIC P50 CA114747 (S.S.G.) and NCI 5RO1 CA082214 (S.S.G.) awarded by the NCI. The government has certain rights in the invention.

### BACKGROUND

Luminescence is a phenomenon in which energy is specifically channeled to a molecule to produce an excited state. Return to a lower energy state is accompanied by release of a photon. Luminescence includes fluorescence, phosphorescence, chemiluminescence, and bioluminescence. Bioluminescence is the process by which living organisms emit light that is visible to other organisms. Where the luminescence is bioluminescence, creation of the excited state derives from an enzyme catalyzed reaction. Luminescence can be used in the analysis of biological interactions.

Luciferases are commonly used as reporter genes, and hopefully in the future as bioluminescent labels, in a variety of biological assays performed both in vitro and in vivo. For in vitro assays such as cell culture transfection studies, the wavelength of light that a luciferase yields is usually of little consequence. For in vivo assays such as small animal imaging studies, the wavelength is important because biological tissues are less attenuating to the red and near-infrared portions of the optical spectrum.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

The patent or application file contains at least one drawing executed in color. Copies of this patent or patent application publication with color drawing(s) will be provided by the Office upon request and payment of the necessary fee.

FIG. 1A is a scheme that illustrates a mechanism of coelenterazine oxidation and light production.

FIG. 1B illustrates embodiments of synthesized bisdeoxycoelenterazine derivatives: acetyl-bisdeoxycoelenterazine (7), O-Boc-bisdeoxycoelenterazine (8), acetoxymethyl-bisdeoxycoelenterazine (9), and pivaloyloxymethyl-bisdeoxycoelenterazine (10).

FIG. 2 illustrates the rate of the bioluminescent reaction for BDC (□), acetyl-BDC (▪), O-Boc-BDC (), acetoxymethyl (▴), and pivaloyloxymethyl (□), evaluated as the change of the maximum light signal over time.

FIG. 3A illustrates the change in luminescence over time for acetyl-bisdeoxycoelenterazine (7), O-Boc-bisdeoxycoelenterazine (8), acetoxymethyl-bisdeoxycoelenterazine (9), and pivaloyloxymethyl-bisdeoxycoelenterazine (10). Error bars represent standard deviation from the average value. FIG. 3B illustrates the bioluminescence imaging of HT1080 cells expressing GFP2-RLUC fusion protein after exposure to bisdexycoelenterazine and its derivatives. Color bar units are p/sec/cm2/sr, where p stands for photon and “sr” stands for steradian.

FIG. 4 illustrates an HPLC chromatogram for acetyl-BDC. The retention time was 9.6 minutes.

FIG. 5 illustrates the absorbance and fluorescence spectra for acetyl-BDC. Maximum absorbance was at 260 nm and maximum fluorescence at 395 nm.

FIG. 6 illustrates an HPLC chromatogram for O-Boc-BDC. Retention time was 12.7 minutes.

FIG. 7 illustrates an absorbance and fluorescence spectra for O-Boc-BDC. Excitation maximum was at 260 nm and emission maximum at 395 nm.

FIG. 8 illustrates an HPLC chromatogram for acetoxymethyl-BDC. Retention time was 8.7 minutes.

FIG. 9 illustrates an absorbance and fluorescence spectra for acetoxymethyl-BDC. Absorption maximum was at 264 nm and fluorescence maximum at 410 nm.

FIG. 10 illustrates an HPLC chromatogram for pivaloyloxymethyl-BDC. The retention time was 11.0 minutes.

FIG. 11 illustrates the absorbance and fluorescence spectra for pivaloyloxymethyl-BDC. Absorption maximum was at 264 nm and fluorescence maximum at 410 nm.

FIG. 12 illustrates a western blot analysis of GFP2-Rluc8 protein in the cells exposed to Rluc substrates for different amount of time.

FIGS. 13A-13D illustrates mammalian expression of the embodiments of the BRET vectors using Renilla luciferase mutants as a donor. FIG. 13A illustrates fluorescent photomicrographs of transiently transfected 293T cells expressing either GFP²-RLUC or GFP²-RLUC8 fusion 24 hours after transfection.

FIG. 13B illustrates semi-quantitative western blot analysis showing donor (RLUC) and acceptor (GFP²) protein expression in 293T cells transiently transfected with donor alone and fusion plasmids as marked. GFP²-Rluc-C, GFP²-Rluc-M, and GFP²-Rluc8 indicate BRET fusions using the single mutation C124A RLUC, double mutation C124A/M185V RLUC, and eight mutations RLUC8 donor respectively. α-tubulin was used as loading control.

FIG. 13C illustrates the same cells as mentioned in FIG. 13B that were plated (10,000/well in 48 well plate) and imaged with a CCD camera after adding equal amount of Clz400 substrate in each well. Mean photon values were determined by drawing ROIs over triplicate samples. The chart represents the normalized mean BRET ratio (bar) and RLUC emission light outputs (line). Error bars represents SEM.

FIG. 13D illustrates semi-quantitative assessment of BRET donor and acceptor proteins by western blotting in selected clonal populations of HT1080 cells expressing the fusion constructs. α-tubulin was used as a loading control. After checking the fusion protein expression in clonal populations, a fixed number of each cell types were plated and within 4 hours CCD camera imaging was performed by adding equal amount of Clz400 in well plates. ROI values from corresponding wells were plotted as obtained from image data using either a donor or acceptor filter. Error bars represents SEM.

FIGS. 14A and 14B illustrates that BRET signal can be spectrally resolved from mammalian cells expressing the BRET fusion vector. FIG. 14A illustrates CCD camera image of a few HT1080 cells stably transfected with Rluc8 and GFP²-Rluc8 plasmid vector from individual wells of a 96 well plate. Cell imaging was done by adding Clz400 substrate (0.5 μg/well) 4 hours after plating. Spectral separation of emission light from individual clonal cells transfected with native Rluc or GFP²-Rluc plasmid was not possible. Pseudocolor scale bar represents luminescence photon output averaged for the three filters. FIG. 14B confirms that the true nature of signals from individual cells, parallel wells containing cells were imaged at 3 and 22 hours after plating. As the cells divide over time, acceptor and donor signal intensities are doubled. Individual cells of the marked ROI locations were photographed using a light microscope after CCD imaging.

FIGS. 15A and 15B illustrates the localization of BRET signal from subcutaneous and deep tissue structures of a nude mouse implanted with cells constitutively over-expressing GFP²-RLUC8. FIG. 15A illustrates a CCD camera image of a representative mouse implanted with 5×10⁵ GFP²-Rluc cells on the left shoulder (L) and the same number of GFP²-Rluc8 cells on the right flank (R). The mice were injected with 25 □g Clz400 substrate via tail-vein and imaged using a 2 minutes image acquisition time. FIG. 15B illustrates a CCD camera image of a representative mouse injected with 2×10⁶ GFP²-Rluc8 cells by tail-vein injection. 30 minutes later the mouse was injected with 75 μg Clz400 and imaged immediately using a 3 minutes acquisition time. Unlike cells that stably express GFP²-RLUC, both donor and acceptor signal from GFP²-Rluc8 expression can be measured from the lungs. For both FIGS. 15A and 15B, images were first captured using the GFP filter followed by the DBC filter after a single injection of Clz400.

FIGS. 16A to 16E illustrate the characterization of a BRET sensor for testing a small molecule dimerizer drug in mammalian cells. FIG. 16A illustrates a diagram showing the BRET vector construct, where two individual mTOR pathway protein sequences (FRB and FKBP12) were cloned between the donor and acceptor molecule using the specified amino acid linkers. FKBP12 and FRB domains dimerize only in the presence of the small molecule dimerizer rapamycin, bringing the acceptor and donor in close proximity. FIG. 16B illustrates that the HT1080 cells constitutively over-expressing the sensor vector were exposed to measured quantities of rapamycin for 20 hours and then the BRET signal was quantitated by imaging with the Clz400 substrate. FIG. 16C illustrates the same cells and they were exposed to 40 nM rapamycin concentration and the BRET signal was measured at various time points after addition of drug. FIG. 16D illustrates a few cells that were plated in a 96 well black well plate and the BRET signal (represented by the line) was determined from individual cells or cells dividing over time, showing that even though the acceptor and donor signal (represented by bars as marked) increases, the BRET ratio remain constant (at a specific drug concentration). FIG. 16E illustrates that the HT1080 cells expressing the GFP²-FRB-FKBP12-RLUC8 fusion were also used to determine the reversible nature of the BRET signal. Positive control (dark dotted line) cells were constantly incubated in media containing 40 nM rapamycin and negative control (light dotted line) cells were incubated in normal media. The experimental cells (solid line) were first incubated in rapamycin (40 nM) containing media for 4 hours, imaged, and then maintained in rapamycin free media until the signal dropped significantly (120 hours scan time point). After imaging at this time point, the cells were re-exposed to rapamycin (40 nM) for 5 hours and imaged again showing increased BRET signal.

FIG. 17 illustrates CCD camera images of individual HT1080 cells constitutively over-expressing the GFP²-FRB-FKBP12-Rluc8 fusion in the presence or absence of rapamycin. FIG. 17 illustrates a 96 well plate containing a few stably selected HT1080 cells expressing the GFP²-FRB-FKBP12-RLUC8 fusion were subjected to different doses of rapamycin as marked and imaged using a CCD camera 4 hours after plating. Individual cells were below detectable threshold with the substrate concentration (0.5 μg/well) and the CCD integration time (1 minute) used. With increasing drug concentration, as the interacting partners dimerize, the BRET partners come in closer proximity, leading to a higher BRET signal and thus enabling detection of BRET specific GFP signal from individual cells. Pseudocolor scale bar represents the average luminescence photon output.

FIG. 18 illustrates Table 1 that describes that following introduction of the mutations, marked increases in the activity (photon output) of the Rluc-M and Rluc8 occurs.

FIG. 19 illustrates the time kinetics of photon yields followed for 10 minutes at each filter and the line curves were drawn to show the fit and for obtaining a decay correction factor at each filter.

### SUMMARY

Embodiments of the present disclosure provide for: compositions, BRET systems, kits, and the like. Embodiments of the composition, among others, include: a BDC derivative represented by structure selected from structure A and structure B as described herein.

Embodiments of the BRET system, among others, include: a Renilla luciferase protein, mutant, variant, or derivative thereof, a fluorescent protein, mutant, variant, or derivative thereof, and a BDC derivative represented by structure selected from structure A and structure B as described herein.

Embodiments of the kit, among others, include: a Renilla luciferase protein, mutant, variant, or derivative thereof, a fluorescent protein, mutant, variant, or derivative thereof, and a BDC derivative represented by structure selected from structure A and structure B as described herein.

These embodiments, uses of these embodiments, and other uses, features and advantages of the present disclosure, will become more apparent to those of ordinary skill in the relevant art when the following detailed description of the preferred embodiments is read in conjunction with the appended figures.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of synthetic organic chemistry, biochemistry, biology, molecular biology, recombinant DNA techniques, pharmacology, imaging, and the like, which are within the skill of the art. Such techniques are explained fully in the literature.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

### DEFINITIONS

In describing and claiming the disclosed subject matter, the following terminology will be used in accordance with the definitions set forth below.

Bioluminescence (BL) is defined as emission of light by living organisms that is well visible in the dark and affects visual behavior of animals (See e.g., Harvey, E. N. (1952). Bioluminescence. New York: Academic Press; Hastings, J. W. (1995). Bioluminescence. In: Cell Physiology (ed. by N. Speralakis). pp. 651-681. New York: Academic Press.; Wilson, T. and Hastings, J. W. (1998). Bioluminescence. Annu Rev Cell Dev Biol 14, 197-230.). Bioluminescence does not include so-called ultra-weak light emission, which can be detected in virtually all living structures using sensitive luminometric equipment (Murphy, M. E. and Sies, H. (1990), Meth. Enzymo 1.186, 595-610; Radotic, K, Radenovic, C, Jeremic, M. (1998), Gen Physiol Biophys 17, 289-308). Bioluminescence also does not include weak light emissions, which most probably does not play any ecological role, such as the glowing of bamboo growth cone (Totsune, H., Nakano, M., Inaba, H. (1993), Biochem. Biophys. Res Comm. 194, 1025-1029). Bioluminescence also does not include emission of light during fertilization of animal eggs (Klebanoff, S. J., Froeder, C. A., Eddy, E. M., Shapiro, B. M. (1979), J. Exp. Med. 149, 938-953; Schomer, B. and Epel, D. (1998), Dev Biol 203, 1-11).

“Bioluminescent donor protein” refers to a protein capable of acting on a bioluminescent initiator molecule to generate bioluminescence.

“Bioluminescent initiator molecule” (e.g., bisdeoxycoelenterazine (BDC) derivatives) is a molecule that can react with a bioluminescent donor protein to generate bioluminescence.

“Fluorescent acceptor molecule” refers to any molecule that can accept energy emitted as a result of the activity of a bioluminescent donor protein, and re-emit it as light energy.

As used herein, the term “organelle” refers to cellular membrane-bound structures such as the chloroplast, mitochondrion, and nucleus. The term “organelle” includes natural and synthetic organelles.

As used herein, the term “non-nuclear organelle” refers to any cellular membrane bound structure present in a cell, except the nucleus.

As used herein, the term “host” or “organism” includes humans, mammals (e.g., cats, dogs, horses, etc.), living cells, and other living organisms. A living organism can be as simple as, for example, a single eukaryotic cell or as complex as a mammal. Typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications. In some embodiments, a system includes a sample and a host. The term “living host” refers to host or organisms noted above that are alive and are not dead. The term “living host” refers to the entire host or organism and not just a part excised (e.g., a liver or other organ) from the living host.

The term “detectable signal” is a signal derived from non-invasive imaging techniques such as, but not limited to, BRET imaging systems or devices (e.g., CCD camera systems). The detectable signal is detectable and distinguishable from other background signals that may be generated from the host. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, or 40% or more difference between the detectable signal and the background) between detectable signal and the background. Standards and/or calibration curves can be used to determine the relative intensity of the detectable signal and/or the background.

The signal can be generated from one or more features of the present disclosure. In an embodiment, the signal may need to be sum of each of the individual signals from one or more features. In an embodiment, the signal can be generated from a summation, an integration, or other mathematical process, formula, or algorithm, where the signal is from one or more features. In an embodiment, the summation, the integration, or other mathematical process, formula, or algorithm can be used to generate the signal so that the signal can be distinguished from background noise and the like.

The detectable signal is defined as an amount sufficient to yield an acceptable image using equipment that is available for pre-clinical use. A detectable signal maybe generated by one or more administrations of the embodiments of the present disclosure. The amount administered can vary according to factors such as the degree of susceptibility of the individual, the age, sex, and weight of the individual, idiosyncratic responses of the individual, the dosimetry, and the like. The amount administered can also vary according to instrument and digital processing related factors.

### General Discussion

Briefly described, embodiments of this disclosure, among others, include bisdeoxycoelenterazine (BDC) derivatives at the carbonyl imidazopyrazinone moiety of the BDC compound, methods of use, bioluminescence resonance energy transfer (BRET) systems (e.g., BRET2™ system), and the like.

Embodiments of the BDC derivatives decay slower than the natural decay (oxidation) of a coelenterazine substrate and offer longer imaging times for BRET2™. In addition, a BRET2™ system (e.g., BRET2™ (GFP2-Rluc)) that utilizes a BDC derivative has a superior spectral separation between energy donor and acceptor, offering higher resolution and higher signal to noise ratio than other BRET systems. Considering that the half-times for protein-protein interactions vary from brief to long, this improvement in signal sustainability greatly expands the utility of BRET2™ in real time protein-protein interaction imaging.

In this regard, embodiments of the present disclosure can be used to detect, study, monitor, evaluate, localize, quantify, and/or screen, biological or cellular events, such as, but not limited to, protein-protein interactions, cellular localization of proteins, protein phosphorylation, cell-cell fusion, interactions of macromolecule delivery vehicle with cells, and the like, inside a host living cell, tissue, or organ, or a host living organism.

In an embodiment, the BDC derivatives can include an ester or ether modification at the carbonyl imidazopyrazinone moiety of the BDC compound. In an embodiment, the BDC derivative is represented by structure A and structure B.

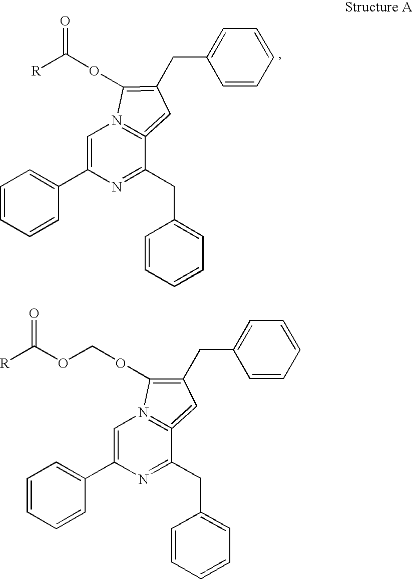

Structure B, wherein R is selected from, but is not limited to, CH₃, CH₃(CH₂)ₙ,

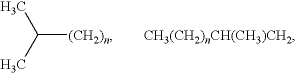

CH₃(CH₂)ₙ(CH═CH)ₙ(CH₂)ₙ, CH₃(CH₂)ₙ(C≡C)ₙ(CH₂)ₙ,

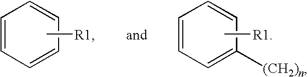

It should be noted that a R1 group could be bonded to one or more of the carbon atoms in the benzene ring. Thus, up to five R1 groups can be attached to benzene ring. Each of the R1 groups can be independently selected from, but are not limited to, electron withdrawing groups, electron donating groups, small alkyl groups (e.g., a methyl group, an ethyl group and larger groups such as a butyl group, a t-butyl group, and the like. The subscript n can be from 1 to 10.

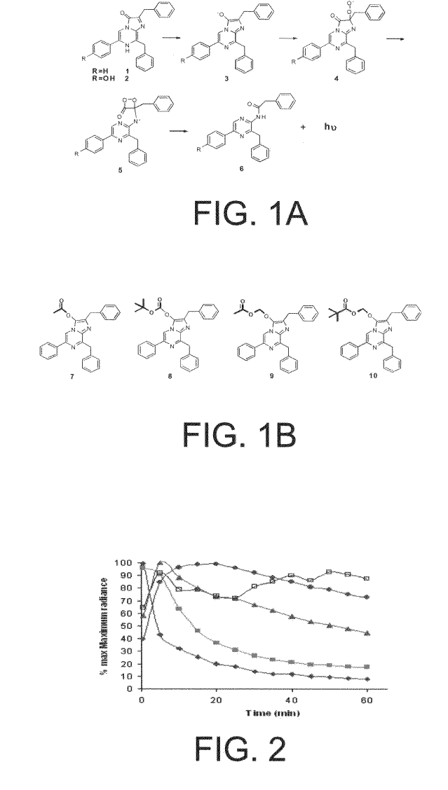

In particular, the BDC derivative includes, but is not limited to, acetyl-bisdeoxycoelenterazine (7), O-Boc-bisdeoxycoelenterazine (8), acetoxymethyl-bisdeoxycoelenterazine (9), and pivaloyloxymethyl-bisdeoxycoelenterazine (10), as shown in FIG. 1B of Example 1.

### BRET System

In general, BRET systems of the present disclosure involve the non-radiative transfer of energy between a bioluminescence donor molecule and a fluorescent acceptor molecule by the FÖRSTER mechanism. The energy transfer primarily depends on: (i) an overlap between the emission and excitation spectra of the donor and acceptor molecules, respectively and (ii) the proximity of about 100 Angstroms (Å) between the donor and acceptor molecules. The donor molecule in BRET produces light via chemiluminescence, so it is amenable to small animal imaging. In addition, the BRET system does not use an external light excitation source, which provides potentially greater sensitivity in living subjects because of the low signal to noise ratio. A signal from the BRET system can be detected with a detection system. The detection system includes, but is not limited to, a light-tight module and an imaging device disposed in the light-tight module. The imaging device can include, but is not limited to, a CCD camera and a cooled CCD camera. Additional details regarding the BRET system, in particular, the BRET2 system, are provided in Examples 1 and 2.

Embodiments of the BRET system can be used to detect (and visualize) and quantitate cellular events in vitro as well as in vivo studies, which decreases time and expenses since the same system can be used for cells and living organisms. Embodiments of the BRET system can test an event occurance in a large number of protein samples, and has the capacity to transition from single cells to living animals without changing the imaging device. In addition, embodiments of the BRET system can be used to detect (and visualize) and quantitate cellular events from a single cell or more.

To date, in most BRET applications, the donor moiety is Renilla luciferase (Rluc) and the acceptor moiety is the yellow fluorescent protein (Xu Y, Piston D W, Johnson C H. A bioluminescence resonance energy transfer (BRET) system: application to interacting circadian clock proteins. Proc Natl Acad Sci USA 1999; 96:151-6, which is incorporated herein by reference for the corresponding discussion). A second system, referred to as BRET2 (Dionne P, Mireille C, Labonte A, et al. BRET2: efficient energy transfer from Renilla luciferase to GFP2 to measure protein-protein interactions and intracellular signaling events in live cells. In: van Dyke K, van Dyke C, Woodfork K, editors. Luminescence biotechnology: instruments and applications. Boca Raton (FL): CRC Press; 2002. p. 539-55, which is incorporated herein by reference for the corresponding discussion), provides for better spectral resolution by using a mutant of the green fluorescent protein (GFP2) as the acceptor and switching the native RLUC substrate, coelenterazine, with the analogue coelenterazine-400a (Clz400; also known as DeepBlueC). GFP2 is an Aequorea victoria GFP mutant adapted for excitation at 400 nm while retaining its 515 nm peak emission. Clz400 is similar to the native substrate in being cell-permeable and nontoxic, but it differs by yielding a 400 nm emission peak rather than the 485 nm (from—De A et al., Cancer Res 2007; 67(15):7175-83, which is incorporated herein by reference).

In an embodiment, the BRET system is a BRET2™ system. The BRET2™ system includes a BDC derivative, Renilla luciferase, mutant, variant, or derivative thereof, and a fluorescent protein (e.g., green fluorescent protein) mutant, variant, or derivative thereof. In an embodiment, the BRET2™ system includes mutated variant of GFP (GFP2) and a modified variant of Rluc. The BDC derivatives are described above and in the Examples. The Renilla luciferase and fluorescent protein (GFP2) can be purchased from PerkinElmer Inc, and are also know in the art. The accession number for Rluc8 is EF446136, and the accession number for Rluc M63501, where each sequence is incorporated herein by reference.

Embodiments of the present disclosure can be used to produce a detectable signal that can be used in imaging to detect, study, monitor, evaluate, localize, quantify, and/or screen, biological or cellular events, such as, but not limited to, protein-protein interactions, cellular localization of proteins, protein phosphorylation, cell-cell fusion, interactions of macromolecule delivery vehicle with cells, and the like, inside a host living cell, tissue, or organ, or a host living organism.

### Kits

Embodiments of the present disclosure includes kits that may include, but are not limited to, a BDC derivative, Renilla Luciferase proteins, fluorescent proteins, and the like, and directions (written instructions for their use). In particular, embodiments of the present disclosure include kits that include a BDC derivative and Renilla Luciferase proteins and fluorescent proteins appropriate for BRET2 systems. The components listed above can be tailored to the particular study to be conducted (e.g., protein-protein interaction). The kit can further include appropriate buffers and reagents known in the art for administering various combinations of the components listed above to the host cell or host organism.

### EXAMPLES

Now having described the embodiments of the present disclosure, in general, examples 1 and 2 describes some additional embodiments of the present disclosure. While embodiments of the present disclosure are described in connection with examples 1 and 2 and the corresponding text and figures, there is no intent to limit embodiments of the present disclosure to these descriptions. On the contrary, the intent is to cover all alternatives, modifications, and equivalents included within the spirit and scope of embodiments of the present disclosure.

### Example 1

Protein-protein interactions are the basis of many important cellular functions. The ability to non-invasively image these and other interactions in living organisms provides a unique possibility of studying important biological processes in intact, minimally perturbed systems. Molecular imaging assays based on bioluminescence resonance energy transfer (BRET) are one of the key strategies for investigating protein-protein and other interactions in live cells as well as in living subjects. BRET is a phenomenon involving a transfer of energy, obtained from the oxidation of substrate by the energy donor, an oxygenase protein, and an energy acceptor, a fluorescent protein. Typically, BRET systems utilize Renilla luciferase (RLuc) as an energy donor, and green fluorescent protein (GFP), and its mutants as an energy acceptor. Based on the specific Renilla substrate utilized, BRET systems are currently divided into BRET1, BRET2 and recently introduced eBRET. Of the three, the BRET2 system has the best spectral separation between the donor and the acceptor, and thus higher signal to background ratio than all possible BRET1 and eBRET combinations. Despite the superior spectral resolution, the BRET2 system has not been applied as broadly and as frequently as BRET1, due to the poor quantum yield and very fast kinetics of oxidation of the substrate utilized, bisdeoxycoelenterazine (BDC 1). The fast oxidation of BDC results in a light signal that decays rapidly, limiting stable signal detection to only a few seconds. We report here BDC derivatives that improve kinetics of BDC oxidation and consequently offer significantly longer lasting light signal. Considering that the half-times for protein-protein interactions vary from brief to long, this improvement in signal sustainability greatly expands the utility of BRET2 in real time protein-protein interaction imaging.

Oxidation of coelenterazine, the natural RLuc substrate, can generate light as a result of both enzymatic and chemical reaction, phenomena termed bioluminescence and chemiluminescence respectively. Although the mechanism of coelenterazine oxidation is thought to be the same (Scheme 1) for both of these reactions, the kinetics of light production differ quite remarkably. Bioluminescent reactions have very fast kinetics, leading to the production of intense, exponentially decaying light signal. On the other hand, chemiluminescence caused by the reaction of coelenterazine with aprotic organic solvents in the presence of oxygen and base results in longer lasting light signal of lower intensity. The rate limiting step in chemiluminescence reactions seems to be the formation of a peroxide intermediate. Coelenterazine peroxide has been synthesized and detected at −80° C. by NMR. At temperatures higher than −50° C., the hydroperoxide intermediate spontaneously decomposes with emission of light. The decrease in the rate of the formation of the peroxide intermediate should therefore result in the decrease of the rate of the bioluminescent reaction and sustained production of light. We achieve this by introducing protecting groups at the reaction site, the carbonyl group of the imidazopyrazinone moiety, that have to be removed before the peroxide can be formed and oxidation can take place. The deprotection of the reaction site can be accomplished by the action of the cellular enzymes such as esterases, which makes the derivatives suitable as live cell substrates.

We have synthesized four BDC derivatives (FIG. 1A) with varying size and type of the groups at the carbonyl imidazopyrazinone moiety. The BDC derivatives shown in FIG. 1B include: acetyl-bisdeoxycoelenterazine (7), O-Boc-bisdeoxycoelenterazine (8), acetoxymethyl-bisdeoxycoelenterazine (9), and pivaloyloxymethyl-bisdeoxycoelenterazine (10).

BDC (1) was synthesized following a published procedure (See, Bioorg. Chem. 2007, 35, 82-111, which is incorporated herein by reference for the corresponding discussion) by coupling 2-amino-3-benzyl-5-phenylpyrazine and 1,1-diethoxy-3-phenylacetone. Subsequent reaction with acetic anhydride, di-tert-butyl dicarbonate, bromomethyl acetate and chloromethyl pivalate, afforded acetyl-bisdeoxycoelenterazine (7), O-Boc-bisdeoxycoelenterazine (8), acetoxymethyl-bisdeoxycoelenterazine (9), and pivaloyloxymethyl-bisdeoxycoelenterazine (10).

The kinetics of the bioluminescent reactions was evaluated by exposing the derivatives to HT1080 cells stably expressing the GFP2-RLuc8 fusion protein and detecting generated light over the course of one hour using cooled charge coupled device camera (FIG. 2). FIG. 2 illustrates the rate of the bioluminescent reaction for BDC (□), acetyl-BDC (▪), O-Boc-BDC (), acetoxymethyl (▴), and pivaloyloxymethyl (□) evaluated as the change of the maximum light signal over time.

The luciferase used in this system is the mutant RLuc8 developed in our lab that in the reaction with BDC gives 59 fold higher light output than the native RLuc16. Of the four derivatives only acetyl-BDC showed fast light signal decay, similar to BDC. Not surprisingly, the acetyl ester seems to be easily cleaved by cellular esterases and the bioluminescent reaction is only slightly altered. The bulkier tert-butyloxycarbamyl group at the carbonyl of the imidazopyrazinone moiety results in the derivative that shows considerably slower kinetics. The peak light emission for O-Boc-BDC was observed 15 minutes after exposure to the RLUC8 expressing cells, and remained fairly stable over one hour. In comparison, at the 15 minute time point, the parent BDC lost nearly 75% of its initial light emission. Bulkiness of the t-butyl group appears to considerably slow down the enzymatic ester hydrolysis, and thus the bioluminescent reaction, providing a longer lasting light signal. The size of the protecting group had the same effect on the rate of the bioluminescent reaction in the case of the ether derivatives, acetoxymethyl-BDC and pivaloyloxymethyl-BDC. Acetoxymethyl group delayed the emergence of the maximum light signal by 5 minutes compared to the parent BDC, after which time the signal slowly decayed with time. Retardation of the enzymatic ether cleavage caused by the bulky t-butyl group in the case of pivaloyloxymethyl-BDC resulted in the slowest bioluminescent reaction. Although the light signal fluctuated with time, it did not fall below 88% of the maximum intensity. In terms of the signal half-life, defined as the time required for the initial light flux to fall to its half-value, compared to BDC, derivatives (8, 9 and 10) have much improved characteristics. Compared to only 5 minutes half-life of the parent BDC, derivative (8) had a half life of ˜50 minutes, while half lives of derivatives (9) and (10), were even longer than one hour.

The rate of the bioluminescent reaction also depended on the type of the protecting group. Derivatives carrying ether protecting groups at the carbonyl of the imidazopyrazinone ring, (9) and (10), showed slower bioluminescent reactions compared to their ester counterparts, derivatives (7) and (8).

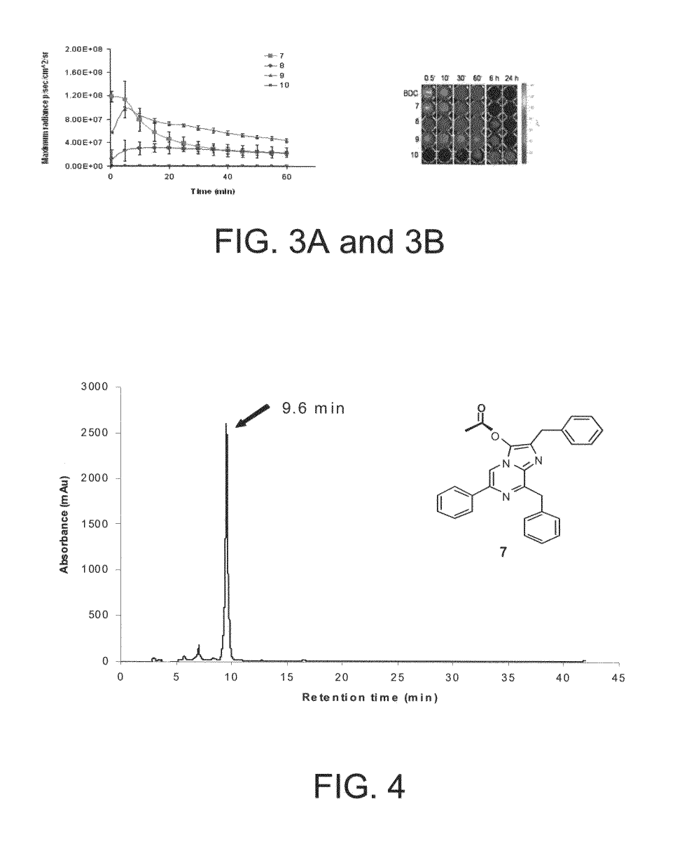

The carbonyl group protection inevitably affects the intensity of the light signal of the derivatives. Of the four derivatives, the highest light signal was observed with acetyl-BDC and the lowest with pivaloyloxymethyl-BDC (FIG. 3A). Although the signal intensity of pivaloyloxymethyl-BDC never reached the intensity of the signal of any of the other three BDC derivatives, the only observable signal 24 hours after exposure to the enzyme came from it (FIG. 3B).

FIG. 3A illustrates the change in luminescence over time for acetyl-bisdeoxycoelenterazine (7), O-Boc-bisdeoxycoelenterazine (8), acetoxymethyl-bisdeoxycoelenterazine (9), and pivaloyloxymethyl-bisdeoxycoelenterazine (10). Error bars represent standard deviation from the average value. FIG. 3B illustrates the bioluminescence imaging of HT1080 cells expressing GFP2-RLUC fusion protein after exposure to bisdexycoelenterazine and its derivatives. Color bar units are p/sec/cm2/sr, where p stands for photon and “sr” stands for steradian.

Clearly, the decrease in the rate of the bioluminescent reaction is closely related to the decrease in signal intensity. It is important to point out here that the lower light signal does not render a BDC derivative inadequate for application in BRET2 assays. Just as in the case of Enduren™, the coelenterazine h derivative with prolonged lifetime, signal intensity can be increased by simply using higher concentrations of the substrate in BRET assays. What makes a derivative have high utility in BRET2 applications is the decelerated kinetics of its oxidation resulting in sustained emission of light.

In summary, protection of the carbonyl group of the imidazopyrazinone moiety in bisdeoxycoelenterazine led to derivatives with improved kinetics of the bioluminescent reaction. Our results indicate that the extent of the effect the protecting group has on the rate of oxidation depends on the size and type of the protecting group. Of the four synthesized BDC derivatives, three (8, 9, and 10) show great promise for improving the existing BRET2 assays in terms of light signal sustainability. The longer lasting signal offers a possibility of real-time imaging of protein-protein interaction in live cells in combination with unparalleled signal to background ratio.

### Additional Discussion Regarding Example 1

### General

With the exception of 2-amino-3-benzyl-5-phenylpyrazine that was obtained from InnoChemie GmbH (Würzburg, Germany), all reagents and solvents were purchased from Sigma-Aldrich Chemical Co. (St. Louis, Mo.) and used without further purification. All 1H NMR spectra were obtained on a Varian XL-400 (Varian, Palo Alto, Calif.) at 400 MHz. Electron spray ionization (ESI) mass spectrometry was done by Vincent Coates Foundation Mass Spectrometry Laboratory, Stanford University. Absorbance spectra were obtained on Agilent UV-visible Chemstation. Fluorescence spectra were recorded on FluoroMax-3 Jobin Yvon fluorometer. Purification and analysis of the products was performed using the Dionex Summit® high-performance liquid chromatography (HPLC) system (Dionex Corporation, Sunnyvale, Calif.), equipped with a 340U 4-Channel UV-Vis absorbance detector. Reverse phase HPLC column Dionex Acclaim® 1120 (C18, 4.6 mm×250 mm) was used for the analysis of the products. The mobile phase was 0.1% trifluoroacetic acid (TFA) in water and 0.1% TFA in acetonitrile (CH₃CN). The flow was 1 mL/min under isocratic conditions of 85% CH₃CN. The products were detected by following absorbance at 260 nm wavelength.

Synthesis of 1,1-diethoxy-3-phenylacetone

Ethylethoxy diacetate (1.78 mL, 10 mmol, 1 eq) was dissolved in 40 mL anhydrous THF and cooled to −78° C. Benzylmagnesium chloride (7.5 mL 2M benzylmagnesiumchloride in THF, 15 mmol, 1.5 eq) was added dropwise, under argon to the reaction mixture. After 2 hours of stirring the reaction was quenched with saturated ammonium chloride. When the reaction mixture warmed to room temperature, it was diluted with 50 mL and the organic products extracted with ethylacetate (3×50 mL). After drying with anhydrous MgSO₄, the solvent was removed on a rotary evaporator and the crude mixture purified by column chromaptography with 10% ethyl acetate in hexane as eluent. Yield 2.04 g (91.8%). 1H NMR (CDCl3, 400 MHz) 8.31 (2H, t, 7.5 Hz) 7.22 (3H, m) 4.63 (1H, s) 3.88 (2H, s) 3.70 (2H, m) 3.54 (2H, m) 1.24 (6H, t, 7 Hz). Calculated molecular weight 222.1. Found ESI+244.8 (M⁺Na⁺).

Synthesis of bisdeoxycoelenterazine (BDC, 1)

2-amino-5-benzyl-1-phenyl-pyrazine (200 mg, 0.765 mmol, 1 eq) and ketoacetal (340 mg, 1.53 mmol, 2 eq) were dissolved in 10 mL ethanol and kept under argon. To the reaction mixture were added 0.4 mL concentrated hydrochloric acid and mixture refluxed overnight. The precipitate that formed was collected and washed with cold ethanol. Yield 215 mg (71.7%). 1H NMR (DMSO, 400 MHz) 8.76 (1H, b) 7.99 (2H, m) 7.49-7.21 (12H, m) 4.56 (2H, s) 4.29 (2H, s). Calculated molecular weight is 391.2. Found (ESI+) 392.2.

Synthesis of acetyl-bisdeoxycoelenterazine (7)

Bisdeoxycoelenterazine (10 mg, 25.6 μmol, 1 eq) was dissolved in 0.5 mL pyridine. Under argon acetic anhydride (23 μl, 0.256 mmol, 10 eq) was added and the reaction mixture stirred for one hour. The solvent was removed in vacuo and crude product purified by column chromatography using ethyl acetate as eluent. Yield 7.4 mg (67.2%). 1H NMR (CDCl₃, 400 MHz) 7.86 (2H, dd, 1.4 Hz, 8.4 Hz) 7.77 (1H, s) 7.61 (2H, d, 7 Hz) 7.46-7.22 (11H, m) 4.62 (2H, s) 4.20 (2H, s) 2.15 (3H, s). Calculated molecular weight is 433.2. Found (ESI+) 434.2.

FIG. 4 illustrates a HPLC chromatogram for acetyl-BDC. The retention time was 9.6 minutes.

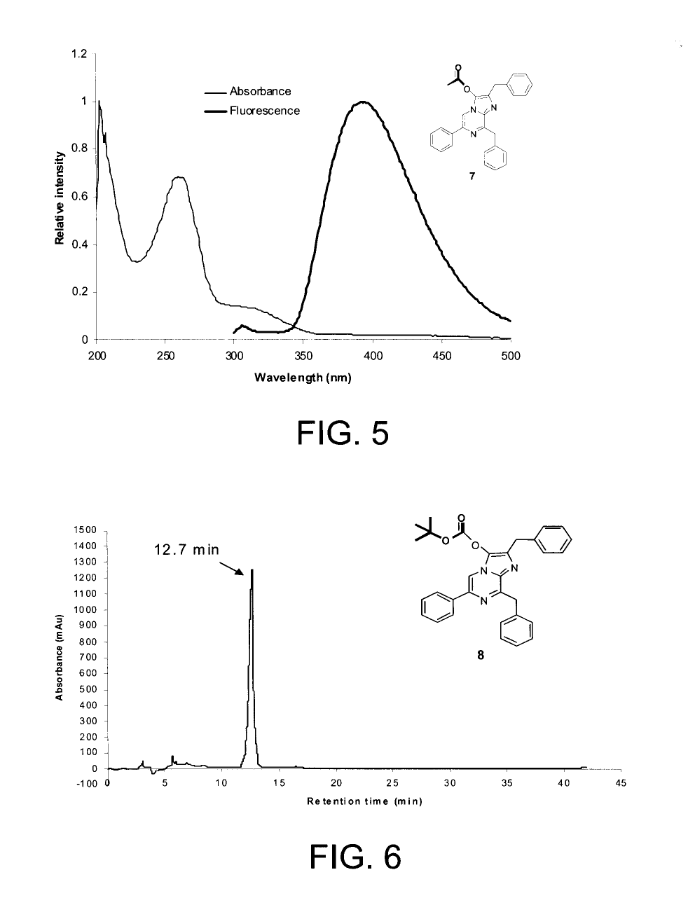

FIG. 5 illustrates the absorbance and fluorescence spectra for acetyl-BDC. Maximum absorbance was at 260 nm and maximum fluorescence at 395 nm.

### Synthesis of O-Boc-bisdeoxycoelenterazine (8)

Bisdeoxycoelenterazine (10 mg, 25.6 μmol, 1 eq) was dissolved in 0.5 mL pyridine. Under argon ditert butyl dicarbonate (59 μl, 0.256 mmol, 10 eq) was added and the reaction mixture stirred for 30 min. The solvent was removed in vacuo and the product isolated by column chromatography with ethyl acetate as eluent. Yield 8.1 mg (65%). 1H NMR (CDCl₃, 400 MHz) 7.89 (3H, m) 7.59 (2H, dd, 1.5 Hz; 8.4 Hz) 7.47-7.22 (10H, m) 4.61 (2H, s) 4.20 (2H, s) 1.51 (9H, s). Calculated molecular weight is 491.2. Found (ESI+) 492.2.

FIG. 6 illustrates a HPLC chromatogram for O-Boc-BDC. Retention time was 12.7 minutes.

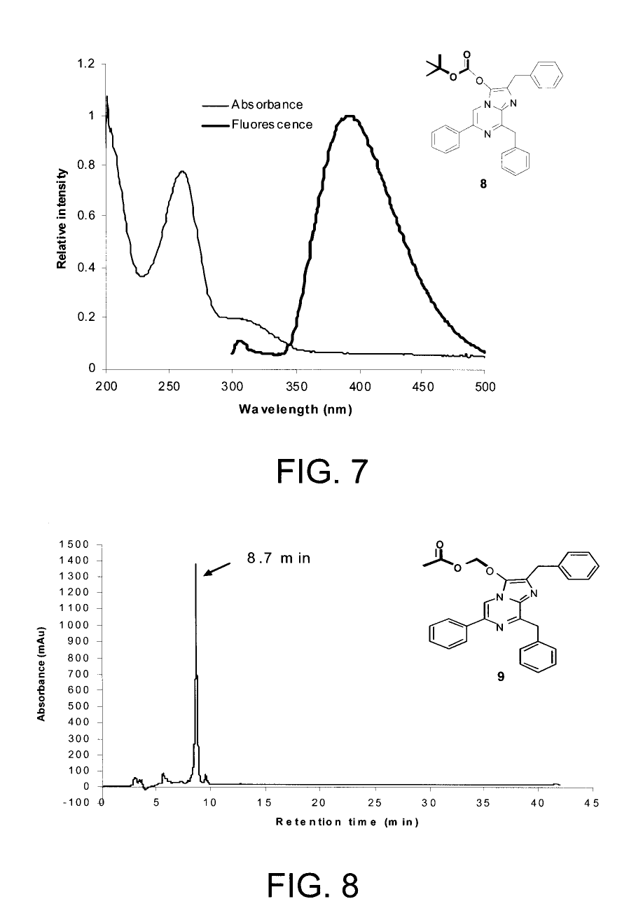

FIG. 7 illustrates the absorbance and fluorescence spectra for O-Boc-BDC. Excitation maximum was at 260 nm and emission maximum at 395 nm.

Synthesis of acetoxymethyl-bisdeoxycoelenterazine (9)

To the mixture of bisdeoxycoelenterazine (10 mg, 25.6 μmol, 1 eq) in 1 mL pyridine were added 13 μL bromomethylacetate (0.13 mmol, 5 eq) and mixture stirred under argon. After usual work-up and purification, 6.6 mg product was isolated (57.6% yield). ¹H NMR (CDCl₃, 400 MHz) 8.13 (1H, s) 7.94 (2H, dd, 1.4 MHz, 8.5 Hz)) 7.62 (2H, d, 7 Hz) 7.46-7.25 (11H, m) 5.43 (2H, s) 4.60 (2H, s) 4.23 (2H, s) 2.01 (3H, s). Calculated molecular weight is 463-2. Found (ESI+) 464.1.

FIG. 8 illustrates a HPLC chromatogram for acetoxymethyl-BDC. Retention time was 8.7 minutes.

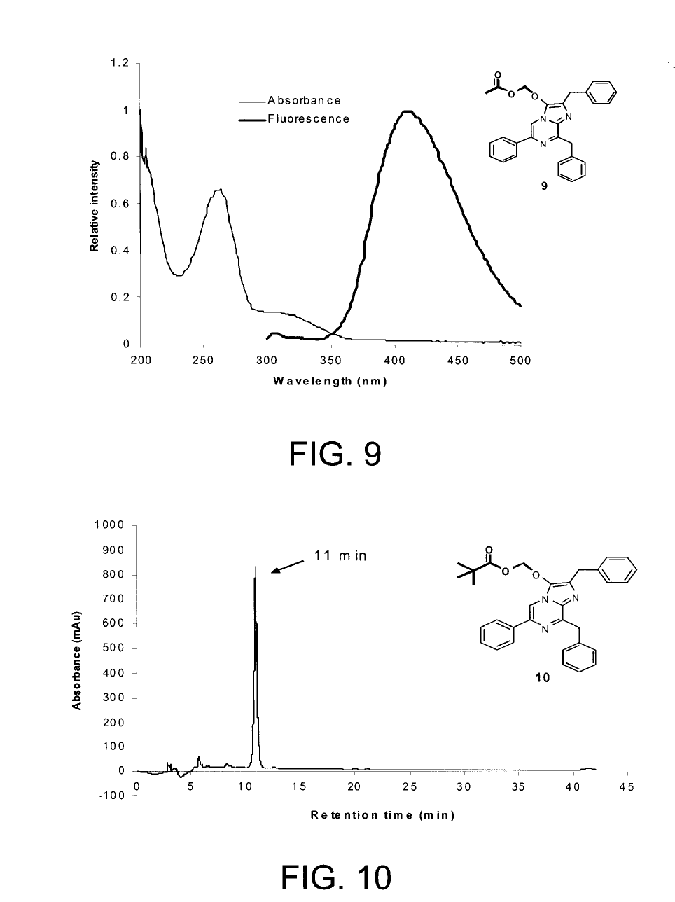

FIG. 9 illustrates the absorbance and fluorescence spectra for acetoxymethyl-BDC. Absorption maximum was at 264 nm and fluorescence maximum at 410 nm.

Synthesis of pivaloyloxymethyl-bisdeoxycoelenterazine (10)

Under argon, to the mixture containing bisdoexycoelenterazine (39.1 mg, 0.1 mmol, 1 eq), potassium carbonate (4.3 mg, 0.03 mmol, 0.03 eq) and chloromethylpivalate (144 μL, 1 mmol, 10 eq) in 400 μL anhydrous DMF was added potassium iodide (17 mg, 0.1 mmol, of KI (17 mg, 0.1 mmol, 1 eq) and mixture stirred overnight. Water was added to the mixture and product extracted with ethyl acetate. Column chromatography with ethyl acetate as eluent afforded 12.5 mg product (24.7%). 1H NMR (CDCl3, 400 MHz) 8.12 (1H, s) 7.92 (2H, d, 7.2 Hz) 7.60 (2H, d, 7.5 Hz) 7.45-7.20 (11H, m) 5.47 (2H, s) 4.60 (2H, s) 4.25 (2H, s) 1.09 (9H, s). Calculated molecular weight is 505.2. Found (ESI+) 506.2.

FIG. 10 illustrates a HPLC chromatogram for pivaloyloxymethyl-BDC. The retention time was 11.0 minutes.

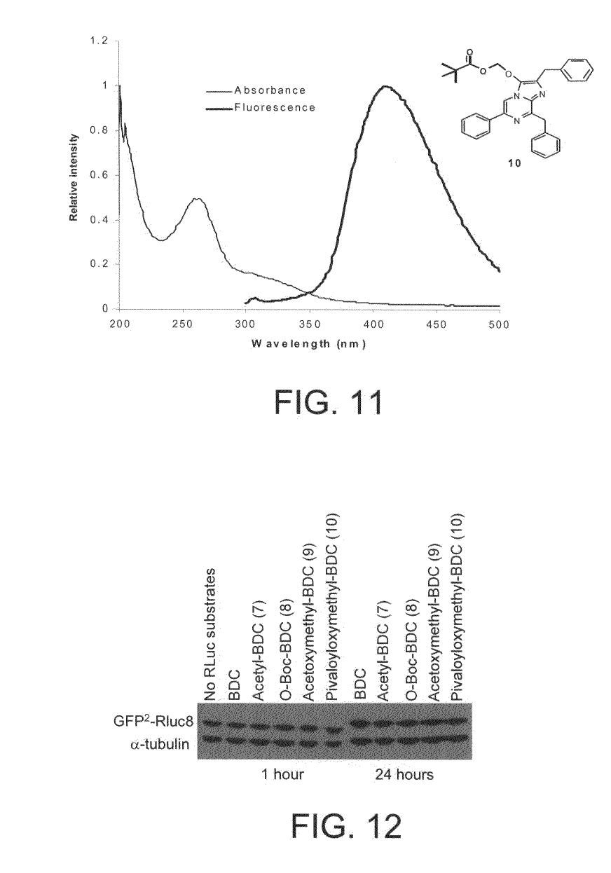

FIG. 11 illustrates the absorbance and fluorescence spectra for pivaloyloxymethyl-BDC. Absorption maximum was at 264 nm and fluorescence maximum at 410 nm.

### Luminescence Measurements

Human fibrosracoma cell line HT1080 stably expressing GFP2-RLuc8 fusion protein was grown in Dulbecco's modified Eagle high glucose medium (DMEM, Invitrogen, Carlsbad, Calif.) supplemented with 10% fetal bovine serum (FBS), 1% penicillin-streptomycin and 500 μg/mL Zeocin™ (Invitrogen). One day before the luminescence study, 10 000 cells were plated in the 96 well plates (Corning Incorporated, Lowell, Mass.). After the removal of the medium 100 μL of the 5 μM solution of BDC in PBS and 60 μM solution of BDC derivatives in DMSO were added and luminescence measured every five minutes during one hour using an open filter setting. The acquisition time was 10 sec. Images were acquired using a cooled charged-coupled device (CCD) camera IVIS 50 (Caliper Life Sciences, Hopkinton, Mass.) and analyzed using Living Image® software version 2.50.1 (Caliper Life Sciences, Hopkinton, Mass.). Region of interest were drawn, and maximum radiance used as the measure of luminescence. The measurements were done in triplicates.

### Western Blot Analysis

Human fibrosracoma HT1080 cells (0.5 million) stably expressing GFP2-RLuc8 fusion protein were plated in the 6 well plates. One day after, the medium was removed and cells exposed to 2 mL 60 μM of BDC and its derivatives in DMEM medium that was not supplemented with FBS. As a control to one of the wells no RLuc substrates were added. After specific time points (1 hour and 24 hours) the medium was removed, cells were washed with ice-cold phosphate buffered saline (PBS) once and lysed in 1× passive lysis buffer (Promega, Madison, Wis.) for 10 minutes on ice. After centrifugation at 13500 rpm for 15 minutes at 4° C., protein content was determined using Bradford assay (Biorad, Hercules, Calif.). 15 μg of total protein were resolved using the 4-12% NU-PAGE gradient gel (Invitrogen, Carlsbad, Calif.) and electroblotted onto nitrocellulose membrane (Schleicher & Schuell, Keene, N.H.). The membrane was first incubated with blocking buffer (5% non fat dry milk in Tris-Buffered saline containing 0.01% Tween 20 (TBST)) for one hour, and then overnight at 4° C. with mouse monoclonal RLuc antibody (Milipore, Billerica, Mass.) 1000 times diluted in blocking buffer. The membrane was washed three times with TBST and incubated with peroxidase conjugated anti-mouse IgG (1:3000 dilution in the blocking buffer) for one hour. As a loading control, the membrane was incubated with the monoclonal anti-human α-tubulin antibody (1:5000 dilution in the blocking buffer). Enhanced chemiluminescent method was used for visualization of protein bands.

FIG. 12 illustrates a Western blot analysis of GFP2-Rluc8 protein in the cells exposed to Rluc substrates for different amount of time.

The results of the protein expression analysis indicate that the light output at different time points depends on the rate of the bioluminescent reaction of a particular substrate and not on the level of GFP2-RLuc8 fusion protein expression. In addition, comparison with the protein level in the cells that have not been exposed to any of the substrates showed that none of the substrates have had any observable effect on the expression of the fusion protein, even at long exposure times.

### Example 2

### Introduction

Bioluminescence resonance energy transfer (BRET) is currently utilized for monitoring various intracellular events including protein-protein interactions in normal and aberrant signal transduction pathways. However, the BRET vectors currently used lack adequate sensitivity for imaging events of interest from both single living cells and small living subjects. Taking advantage of the critical relationship of BRET efficiency and donor quantum efficiency, we report generation of a novel BRET vector by fusing a GFP² acceptor protein with a novel mutant Renilla luciferase donor selected for higher quantum yield. This new BRET vector shows an overall 5.5-fold improvement in the BRET ratio thereby greatly enhancing the dynamic range of the BRET signal. This new BRET strategy provides a unique platform to assay protein functions from both single live cells as well as cells located deep within small living subjects. The imaging utility of the new BRET vector is demonstrated by constructing a sensor using two mTOR pathway proteins (FKBP12 and FRB) that dimerize only in the presence of rapamycin. This new BRET vector should facilitate high-throughput sensitive BRET assays including studies in single live cells and small living subjects. Applications will include anti-cancer therapy screening in cell culture and in living small animals.

Interactions of proteins are critical for many biological processes including signal transduction. Signal transduction frequently involves many regulatory proteins that enhance cell proliferation in response to extra-cellular stimuli and therefore aberrant mutations in these regulatory proteins are often potential targets for cancer management. An example of one such regulatory network is the mammalian TOR (mTOR) signaling pathway. Deregulation of this pathway is shown to have a profound effect in diverse human diseases including cancer, and small molecules (rapamycin and its analogs) that target mTOR pathway proteins are attractive therapeutic candidates with increasing clinical interest. In the scenario where either the genetic mutations or the anti-proliferative agents demand rapid screening procedures, optical reporter based functional imaging assays would be ideal. Currently, assays to identify and characterize these interactions are primarily in vitro-binding assays. In the past five years, imaging strategies based on yeast two hybrid assays, reporter complementation assays, and resonance energy transfer (RET) based assay methods have been developed. But all of these approaches have encountered shortcomings limiting their potential to serve as a single imaging assay for measuring protein-protein interactions from both single cells as well as physiologically relevant living small animal models.

In the context of imaging oncogenic cellular events from small animal models, bioluminescence approaches have the potential to be much more sensitive than similar fluorescent or radionuclide based approaches. Several adaptations of bioluminescence imaging have already been devised by our lab and others to detect protein functions and protein interactions in small living animals, such as the inducible luciferase yeast two hybrid system, luciferase complementation, and more recently bioluminescence resonance energy transfer (BRET). Although these approaches show promise in detecting signal from specific protein-protein interactions within small animal subjects, their sensitivity to measure such events from single live cells and from deep tissues within animals is limited. Due to the fact that the emission from luciferases usually yield very low levels of light, counting sufficient photons to estimate brightness from a small area typically requires long acquisition times, thus limiting existing techniques in achieving single cell sensitivity. This single cell sensitivity may be particularly important if one wants to study heterogeneous behavior of individual cells instead of being limited to studying the bulk behavior of groups of cells.

BRET is an emerging, non-destructive, cell-based assay technique that allows detection of protein interactions in real time, thus providing a new window for various proteomics applications including receptor-ligand interactions and mapping of signal transduction pathways etc. This technique is based on a non-radiative energy transfer between two fusion proteins, with one protein containing a bioluminescent moiety as an energy donor, and the other protein a fluorescent moiety serving as the energy acceptor. To date, in most BRET applications, the donor moiety is Renilla luciferase (Rluc) and the acceptor moiety is the Yellow Fluorescent Protein (YFP). A second system, referred to as BRET², provides for better spectral resolution by utilizing a mutant of the Green Fluorescent Protein (GFP²) as the acceptor and switching the native RLUC substrate, coelenterazine (Clz), with the analog coelenterazine-400a (Clz400, also know as DeepBlueC). GFP² is an Aequorea victoria GFP mutant adapted for excitation at 400 nm while retaining its 515 nm peak emission. Clz400 is similar to the native substrate in being cell-permeable and non-toxic, but it differs by yielding a 400 nm emission peak rather than the 485 nm peak of the native substrate.

In this study, we describe development of new BRET vectors by fusing a mutated Renilla donor protein with the GFP² acceptor to achieve a significantly higher BRET efficiency. The new vector is capable of imaging BRET signal from live single cells as well as from superficial and deep tissue structures of small animal models while using a cooled CCD camera based spectral imaging technique. Furthermore, by incorporating a sensor within the new BRET vector based on rapamycin dependent interacting partners from the mTOR pathway, we tested the utility of the system for imaging small molecule dimerizer drug efficacies from intact living single cells.

### Materials and Methods

Materials: pGFP²-Rluc, phRluc-N and pGFP²-C plasmids were from Perkin Elmer. Coelenterazine-400a (Clz400) was from the Molecular Imaging Products Company, Ann Arbor. Zeocin, Geneticin, and all cell culture media was from Invitrogen. Superfect transfection reagent was from Qiagen. 10% Tris-HCl ReadyGels were from Bio-Rad. Renilla luciferase monoclonal antibody (mAb 4400) was from Chemicon, Living color A.V. peptide antibody was from Clonetech, and α-tubulin monoclonal antibody was from Sigma. BRET² specific 370-450 nm (donor) and 500-570 nm (acceptor) filters were from Chroma. Black box CCD imaging systems (IVIS100 or IVIS200) were from Caliper (formerly Xenogen, Alameda, Calif.). 3-4 weeks old nude mice (nu/nu) were from Charles River laboratory.

Plasmid construct: pBRET² (pCMV-GFP²-MCS-Rluc) was used as the template for making BRET vectors. Fusion constructs were made by cloning either single mutation C124A Rluc, double mutation C124A/M185V Rluc, or Rluc8 to replace the Rluc donor sequence (these RLuc variants are described below). The two mTOR pathway proteins, FKBP12 and single FRAP binding domain (FRB) were PCR amplified and cloned using suitable restriction enzyme sites from the multiple cloning site of the control vector. All products were checked by sequencing. All clonal selections were performed on bacto-agar plate with zeocin.

Western blotting: Expression of fusion constructs were verified in mammalian cells using 293T or HT1080 cells. 24 hours post-transfection cells were harvested and lysed on ice using cell lysis buffer (Cell signaling). Equal amount of lysates were ran on 10% Tris-HCl ReadyGels and transferred onto nitrocellulose membrane (Amersham) with a semi-dry blotting system. The blots were probed with either Renilla antibody or with Living color antibody to detect RLUC or GFP² respectively. The α-tubulin antibody was used as loading control.

Cell Culture, Transfection, Clonal Isolation and Luciferase Assay: Human 293T embryonic kidney cells (ATCC, Manassas, Va.) were grown in MEM supplemented with 10% FBS and 1% penicillin streptomycin solution. The HT1080 human fibrosarcoma cells obtained from ATCC were grown in DMEM (high glucose) supplemented with 10% FBS and 1% penicillin streptomycin. Fixed numbers of cells were plated in 24-well plates in normal growth media. Transient transfection was done 24 hours later using Superfect reagent. Each transfection mix consisted of 1 μg of experimental plasmid along with 0.1 μg of pCMV-Fluc plasmid as the transfection control. Stable HT1080 cells expressing pRluc8 were selected with 500 μg/ml geneticin, and for pGFP²-Rluc, pGFP²-Rluc8 and pGFP²-FRB-FKBP12-Rluc8 plasmids with 350 μg/ml zeocin. Cells with highest expression were judged by measuring RLUC activity using the substrate coelenterazine.

In vitro BRET² Assay: For BRET imaging and ratiometric calculations, the cells were seeded in equal number (typically 10000 cells/well unless otherwise mentioned) in 48 well plates, 4-6 hours later the cells were washed with Dulbecco's phosphate buffered saline (D-PBS), with 50 μl fresh D-PBS then added. Just before CCD imaging, 50 μl of diluted Clz400 (0.75 μg/well final concentration in 48 well format) was added and the plates were placed inside the black box CCD imaging (either IVIS100 or IVIS200). All scans were performed in luminescent mode using the sequential image acquisition feature, with 1 minute integration times, a binning of 5, and a field of view (FOV) set at 15 cm, unless otherwise mentioned. Photon outputs were measured using a 500-570 nm and a 370-450 nm band pass emission filter for GFP² and RLUC-Clz400 signal measurement, respectively. An hour later, FLUC signal was collected from individual wells by adding 0.1 μg D-luciferin substrate per well. For single cell imaging the FOV was set to 4 cm by raising the platform. Images were analyzed using LIVING IMAGE v2.5 software (Caliper). For quantitation, regions of interests (ROI) were drawn over the respective signals as visualized on the overlay image and mean average radiance (photons/sec/cm²/steradian) was computed using the software tools.

Animal Imaging of BRET² Expression by Using a Cooled CCD Camera: An aliquot of 0.5×10⁶ HT1080 cells constitutively over-expressing either pGFP²-Rluc or pGFP²-Rluc8 was injected s.c. in a set of 4 nude mice anesthetized with ketamine:xylazine (4:1). One hour after cell injection, Clz400 (25 μg/mouse) diluted in sterile D-PBS (100 μl total volume) was injected via tail vein (i.v.) and the mice were then imaged immediately. Mice were scanned using first the GFP² and then the RLUC filter, with 2 minute acquisitions each. For the deep tissue signal detection experiment, 2×10⁶ pGFP²-Rluc8 expressing cells were injected via tail vein in a set of 5 anesthetized nude mice and a scan was performed half an hour later, using a 75 μg/mouse injection of Clz400. The animals were placed supine in a light-tight chamber, and a gray-scale photographic reference image was obtained under low-level illumination. Photons emitted from implanted cells on mice were collected for 3 minutes using specified filter sets.

Statistical Testing Average radiance values were obtained from both cell culture assays and in vivo mouse experiments by drawing ROIs on the images. These values were used for BRET² ratiometric calculations. All cell culture and mouse group comparisons were performed with the two-sided Student's t test using Microsoft EXCEL. Values of p≦0.05 were considered statistically significant.

### Results

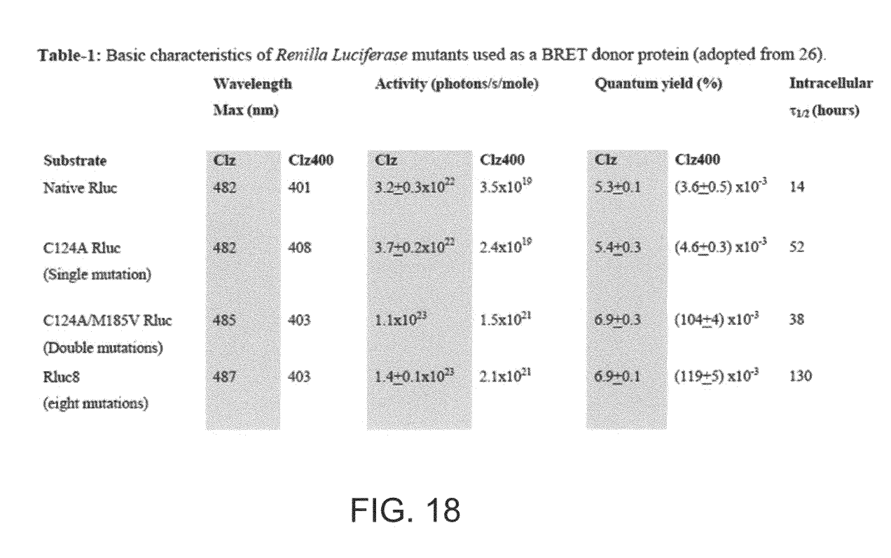

Quantum efficient mutant donors show significant improvement in the BRET efficiency: Generation and basic characterization of Rluc mutations were recently described by our lab (Protein Eng Des Sel 2006; 19:391-400, incorporated herein by reference for the corresponding discussion). We tested three of these mutant variants: 1) single mutation C124A for increased stability (referred to as Rluc-C); 2) a double mutation C124A/M185V for both increased stability and high quantum yield (referred to as Rluc-M) and 3) a combined eight mutations Rluc8 (incorporating A55T, C124A, S130A, K136R, A143M, M185V, M253L, and S287L point mutations) with markedly increased stability and even higher quantum yield than Rluc-M. These three mutant donors had been compared with the native Rluc for their luminous properties while using the native coelenterazine (Clz) substrate and coelenterazine-400a (Clz400). As shown in the Table 1 in FIG. 18, following introduction of the mutations, marked increases in the activity (photon output) of the Rluc-M and Rluc8 occurs. The most characteristic changes are found while measuring the quantum yield of RLUC-M and RLUC8 with Clz400, indicating a key role of the M185V mutation for better utilization of the Clz400 substrate. Whereas, RLUC-M and RLUC8 show only a 1.3-fold improvement in quantum yield compared to the native luciferase with Clz, with Clz400 this increase in quantum yield is ˜28 and ˜32-fold, respectively. It was also noticed that in these mutants, the spectral properties are well maintained with a peak at around 482±5 nm with Clz and around 400±5 nm with Clz400. In addition, for applications that require long term monitoring of protein interactions, significant intra-cellular stability of RLUC8 should be advantageous for its use as a BRET donor.

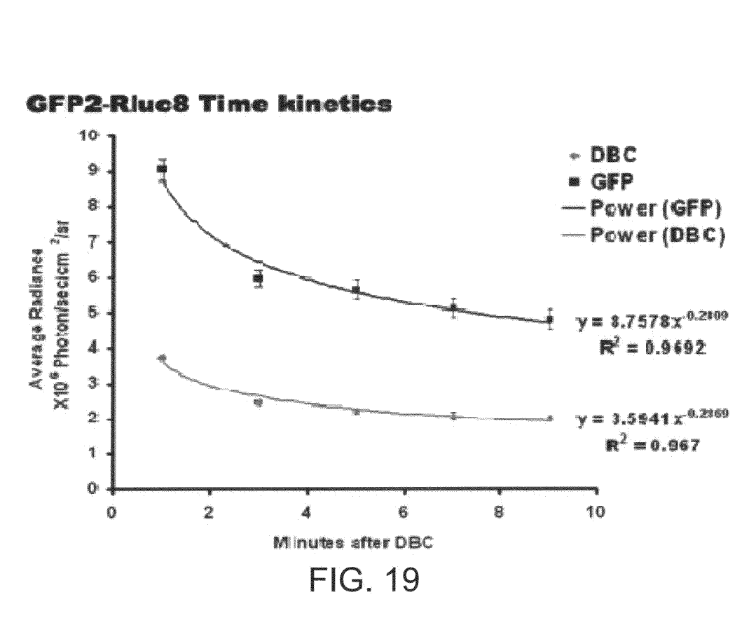

FIG. 19 illustrates the time kinetics of photon yields followed for 10 minutes at each filter and the line curves were drawn to show the fit and for obtaining a decay correction factor at each filter.

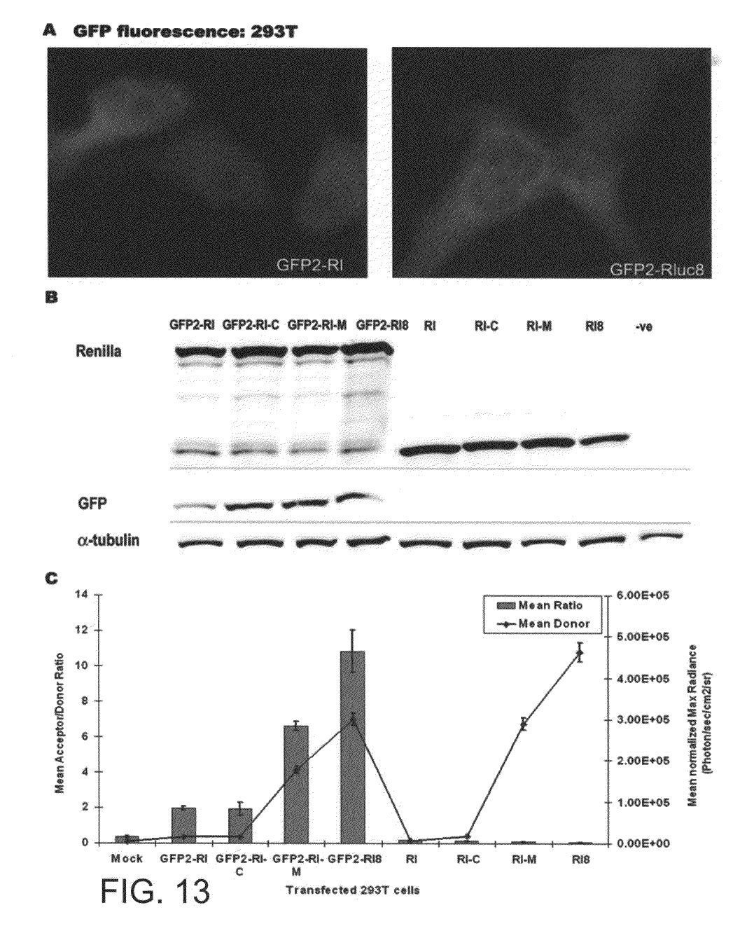

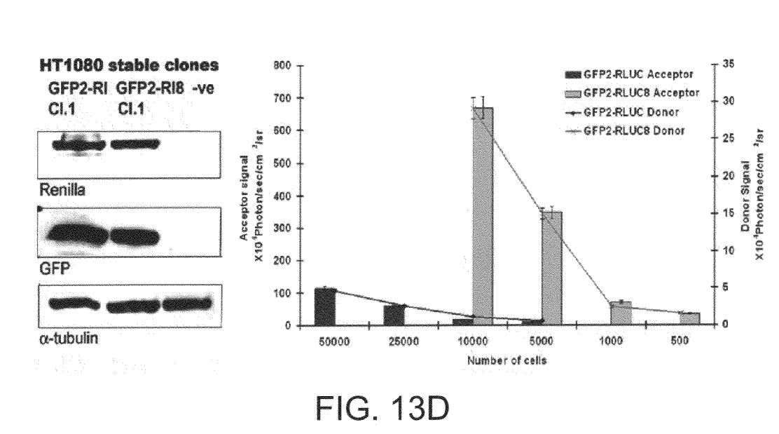

To determine if donors with better quantum efficiencies could contribute in achieving better acceptor output, fusion plasmids were made by replacing native Rluc sequence with the mutated donor sequences. The plasmids, including the linker (Ser-Gly-Ser-Ser-Leu-Thr-Gly-Thr-Arg-Ser-Asp-Ile-Gly-Pro-Ser-Arg-Ala-Thr SEQ ID NO: 1), are otherwise identical to the pGFP²-Rluc plasmid vector (FASEB J. 2005; 05-4628fje, which is incorporated herein by reference). The derived plasmid vectors incorporating Rluc-C, Rluc-M and Rluc8 mutation sequences are referred to as pGFP²-Rluc-C, pGFP²-Rluc-M and pGFP²-Rluc8, respectively. Both GFP fluorescence intensity and semi-quantitative western blot analysis shows equivalent expression of the intact fusion protein in 293T cells transiently transfected with pGFP²-Rluc and pGFP²-Rluc8 (FIG. 13A-13B). However, in comparison to the cells expressing GFP²-RLUC, the normalized donor and acceptor signals obtained by adding the Clz400 substrate show a 35 and 80-fold higher signal for the GFP²-RLUC8 fusion and 25 and 40-fold higher signal for GFP²-RLUC-M, respectively. Following transfection of all the fusion plasmids along with the donor alone plasmids (e.g., pRluc, pRluc-C, pRluc-M, pRluc8) in 293T cells, CCD imaging shows a 3.4 and 5.5-fold increase in the BRET ratio following the characteristic gain in donor signal from RLUC-M and RLUC8 mutant donor (FIG. 13C). Light emission by proteins expressed from plasmids encoding only the donor is helpful in determining the bleed through photons with the GFP² filter, which was ≦5% for all constructs. Based on these results, it is clear that the pGFP²-Rluc8 vector results in the highest gain in the dynamic range of the BRET signal, and this construct was therefore selected for further studies.

Mammalian cells constitutively over-expressing the GFP²-RLUC8 fusion yield about 30-fold higher acceptor light signal than the GFP²-RLUC fusion: HT1080 human fibrosarcoma cells constitutively over-expressing either GFP²-RLUC or the GFP²-RLUC8 fusion protein were established. Isolated clones were judged based on semi-quantitative western blotting results for equivalent expression of each fusion protein (FIG. 13D). After plating varying numbers of each cell type, CCD imaging was performed following addition of equal amounts of Clz400 substrate. FIG. 13D shows that the component light signals increase proportionately with cell number. By comparing the acceptor signal between the two cell lines, it was determined that every GFP²-RLUC8 cell yields a signal equivalent to 30 GFP²-RLUC expressing cells. By comparing the donor signal, each GFP²-RLUC8 cell yields signal equivalent to about 24 GFP²-RLUC cells. By applying decay correction to the values of donor signal (see supplementary data) to correct for signal decay due to the time lapse during scanning, we determined the BRET ratio as 8.6±2.2 for individual cells (n=5) expressing GFP²-RLUC8.

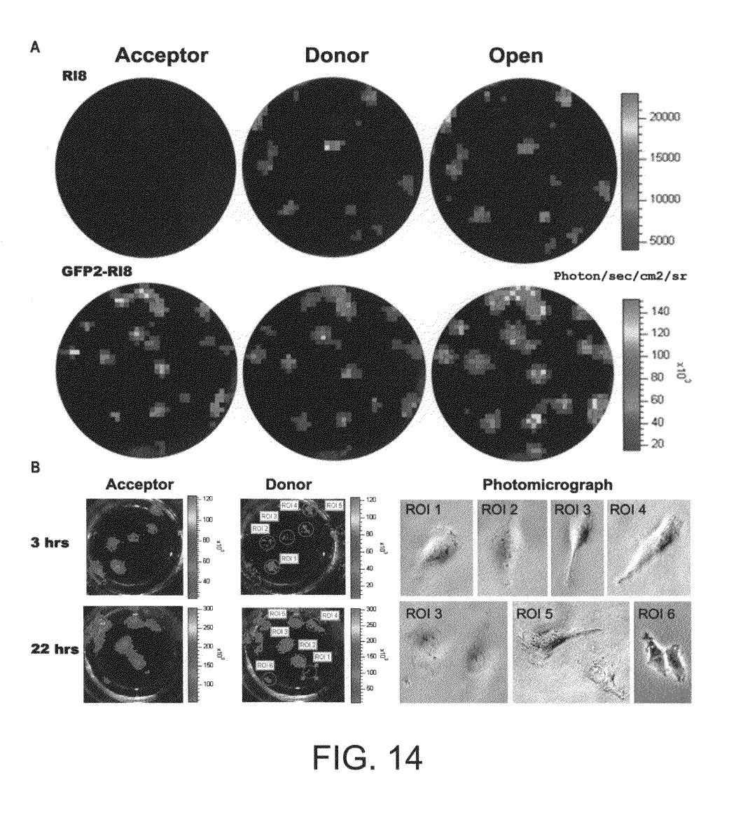

Optical CCD camera imaging can spectrally resolve component light signals as a measure of the BRET signal from individual live cells constitutively over-expressing the GFP²-RLUC8 fusion: To address the capability of BRET measurement from single cells, imaging was performed with the established HT1080 cells constitutively over-expressing either pGFP²-Rluc, pRluc8, or pGFP²-Rluc8 within a few hours after plating. Previously, we reported that BRET² component signals can be resolved from about 30 cells transiently transfected with pGFP²-Rluc plasmid (FASEB J. 2005; 05-4628fje, incorporated herein by reference for the corresponding discussion). Independent of cell types, this vector does not produce enough signal to allow individual cells stably over-expressing the GFP²-RLUC fusion to be resolved, even with the aid of one of the most sensitive cooled CCD camera imaging system available (IVIS 200). Our results here with cells stably expressing RLUC8 as well as the GFP²-RLUC8 fusion (FIG. 14A) show that when the total light output (open filter) is a combination of two major wavelengths of light, CCD imaging can spectrally resolve and quantify the component light signals from individual cells containing these new vectors. For individual cells expressing GFP²-RLUC8, the average radiance with the GFP² filter is 2.5±0.3×10⁴ photon/sec/cm²/sr at 1 minute and with the RLUC filter is 0.6±0.1×10⁴ photon/sec/cm²/sr at 3 minute following addition of Clz400. The photon value obtained with an open filter image of the same cells captured at 5 minute is nearly the same as the sum of the two component signals.

Further evaluation of single cells was performed by allowing cells to grow in culture and then imaging at different time points with equal amount of Clz400 substrate (FIG. 14B). As shown in the figure, selected region of interest (ROI) locations were further documented by observing these locations using a light microscope. The average photon value for well isolated individual cells at the 3 hour time point is 2.8±0.3×10⁴ photon/sec/cm²/sr and 0.5±0.1×10⁴ photon/sec/cm²/sr with acceptor and donor filters, respectively, which doubled (7.1±0.5×10⁴ and 1.3±0.9×10⁴ photon/sec/cm²/sr, respectively) as the cells divide at 22 hours. As these values are well above background (0.4±0.03×10⁴ photon/sec/cm²/sr) (p<0.05), we reasoned that this approach can be extended to studies monitoring BRET signal from specific protein-protein interactions in live individual cells.

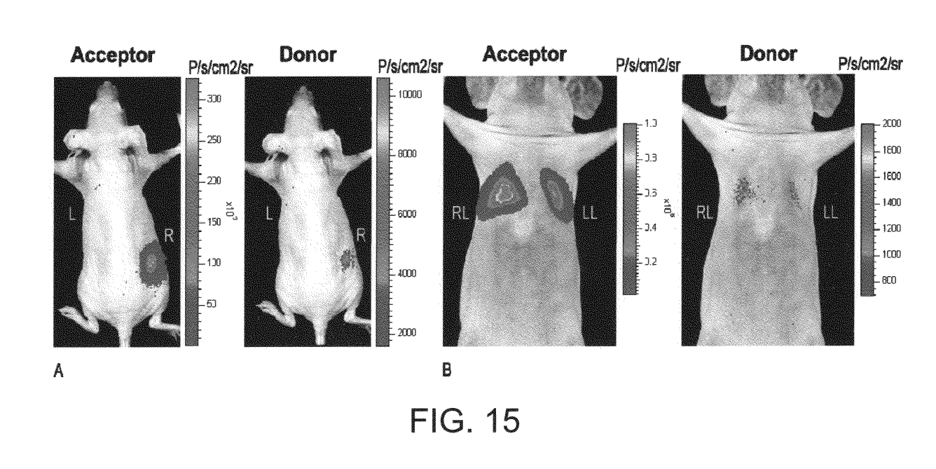

The RLUC8 mutant donor signal can be non-invasively monitored in real time from superficial as well as deeper tissues in living mice: A comparison was performed by implanting 5×10⁵ HT1080 cells over-expressing either pGFP²-Rluc or pGFP²-Rluc8 plasmids in the same mouse. The average radiance from pGFP²-Rluc8 expressing cells yields 49±8×10³ and 1.7±0.2×10³ photon/sec/cm²/sr with the acceptor and donor filters, respectively. This is significantly (p<0.05) higher than the values of 0.6±0.2×10³ and 0.9±0.1×10³ photon/sec/cm²/sr, respectively, obtained from pGFP²-Rluc cells (FIG. 15A). The photon values obtained from stable HT1080 cells expressing GFP²-RLUC are close to the background value (0.3±0.5×10³) and therefore the minimum detectable numbers of these cells should be more than 5×10⁵. Further, both GFP and RLUC component light signals from greater tissue depths were demonstrated to be detectable from lungs by injecting a minimum of 2×10⁶ HT1080 cells over-expressing GFP²-RLUC8 via tail vein followed by an increased amount of Clz400 substrate injection (FIG. 15B). The average radiance of GFP signal from cells that are trapped in the lungs is 22.4±0.8×10⁴ photon/sec/cm²/sr, in comparison to a background value from the lower abdomen of 0.38±0.03×10³ photon/sec/cm²/sr. By turning the filter wheel, the RLUC8 signal collected in the subsequent minutes yield average radiance as 0.37±0.1×10³ photon/sec/cm²/sr and 0.42±0.06×10² photon/sec/cm²/sr for the donor and open filters, respectively. These results indicate that BRET specific acceptor signal can be detected from even a lower number of cells, but due to increased tissue attenuation of shorter wavelength light, donor signal quantitation needed to obtain a true BRET ratio measurement may be limited in small living subjects at greater depths.

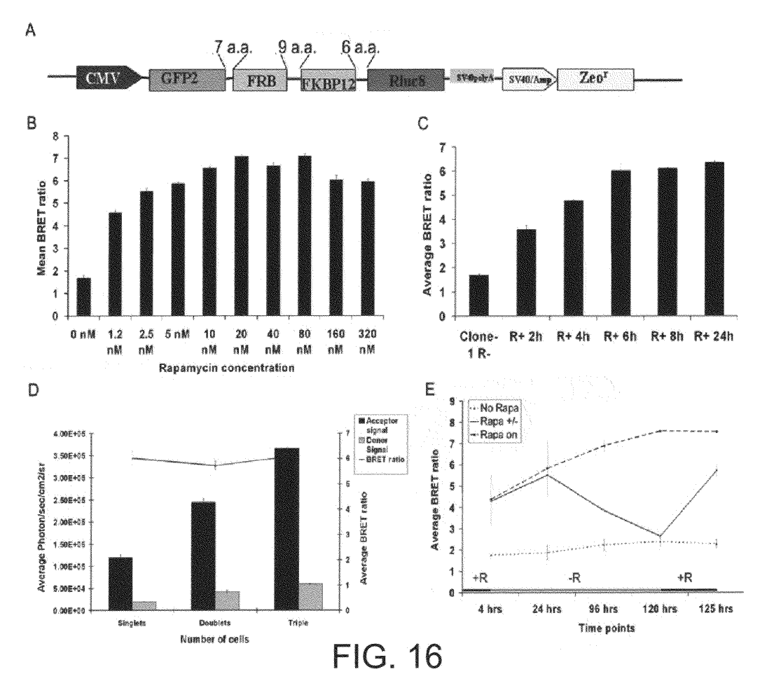

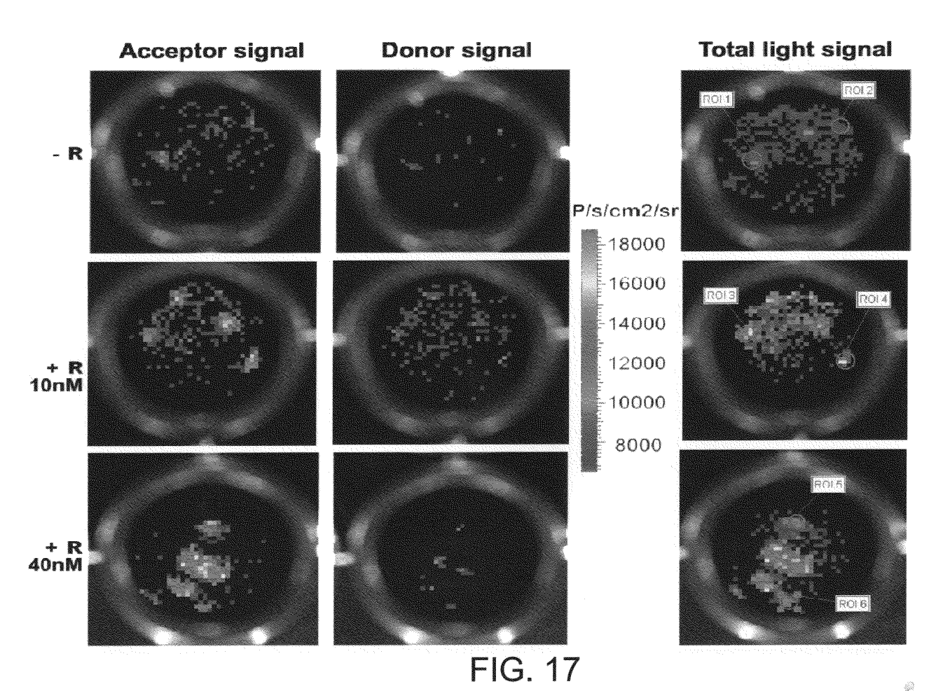

A GFP²-RLUC8 BRET sensor with FKBP12 and FRB as interacting partners can detect rapamycin mediated heterodimerization in vivo at picomolar drug concentrations: To demonstrate the advantage of the new BRET vector, we designed a single vector sensor construct to measure rapamycin mediated dimerization of the two mTOR pathway proteins FKBP12 and FRB. Previously, we have observed that the FRB domain fused to the C-terminus of GFP² and FKBP12 fused to the N-terminus of RLUC successfully shows BRET signal as a result of FKBP12 and FRB interaction in the presence of rapamycin (FASEB J. 2005; 05-4628fje, incorporated herein by reference for the corresponding discussion). Based on that observation, a fusion construct was made by placing FRB and FKBP12 sequences in the linker region of the pGFP²-Rluc8 plasmid as shown in FIG. 16A. HT1080 cells stably over-expressing the vector were used for an imaging based BRET assay. At first, the rapamycin dose response results (FIG. 16B) show that a significant (p<0.05) increase in the BRET signal can be obtained between 1 nM and 80 nM of rapamycin, with a peak ratio of 6.8 at 40 nM. Next, the BRET ratio was determined by exposing cells to 20 nM rapamycin at various time points (FIG. 16C). Starting from a basal BRET ratio of 1.7 for cells incubated without rapamycin, the ratio increased significantly (p<0.05) to a value of 6.1 at 8 hours. A few cells were plated and once these cells settled in isolation, they were exposed to 40 nM rapamycin and imaged over time, showing that even though the growing number of cells at multiple locations show donor and acceptor signal increments, the BRET ratio remain constant independent of cell number (FIG. 16D). By exposing or withdrawing rapamycin from the culture media, we attempted visualization of the reversible nature of the BRET signal (FIG. 17A). On exposure of cells to 40 nM rapamycin for 4 hours, a BRET ratio of 4.4 is observed. As rapamycin is withdrawn and cells are maintained in a rapamycin free environment, the signal drops significantly over 120 hours, with the BRET ratio dropping to 2.65. When the cells are re-exposed to 40 nM rapamycin, a BRET value of 5.7 is observed, which is significantly above (p<0.05) the value of 1.8 for cells never exposed to rapamycin.

Rapamycin mediated dimerization of stably over-expressing heteromeric proteins can be measured from single cells: To evaluate the utility of the current BRET sensor for rapid screening of drugs from a minimal number of cancer cells, we attempted assessment of protein functions by measuring the BRET signal from individual cells (FIG. 17B). Stably selected HT1080 cells over-expressing the GFP²-FRB-FKBP12-RLUC8 fusion protein were plated in isolation and exposed to different concentrations of rapamycin. The cells maintained in culture without rapamycin do not have interaction of the FRB and FKBP12 domains, therefore the acceptor and donor moieties are further apart resulting in only background signal. With increasing rapamycin concentrations, greater numbers of FRB and FKBP12 domains interact, with the result of the interaction being a conformational change of the fusion protein bringing the acceptor and donor moieties in closer proximity. This leads to the significant increase of the acceptor signal to levels above background. We predict that this strategy should be useful for rapid screening of chemicals compounds that function as modulators in various cellular protein networks.

### Discussion

The use of BRET has the potential to significantly increase our understanding of cellular protein networks, especially as this methodology is further improved. In addition to taking advantage of advanced cooled-CCD detector based systems, further improvements in BRET technologies are currently under active investigations to achieve higher sensitivity and suitability for measurements in physiologically relevant model systems. In this study, for the first time, a BRET system is described that can assess protein interactions with high sensitivity from both live individual cells and small living animals. In brief, we have tested donor contributions to the well known GFP²-RLUC (BRET²) systems by altering the native Renilla luciferase sequence with mutations known to increase stability and quantum yield. The eight mutations leading to RLUC8 have greatly improved the donor contribution to the acceptor moiety, thereby increasing the overall sensitivity of the system. This new BRET fusion should be useful for increasing the overall sensitivity of the BRET system, irrespective of the measurement instrument used, leading to either shorter acquisition times and/or allowing use of lower substrate concentration and thus minimizing errors in ratiometric calculations and dependence on decay correction factors of the donor light.

Conceptually, the Förster distance (R₀) (29) is calculated based on the following equation:

R₀=2.11×10⁻²·[κ²·J(λ)·η⁻⁴·Q_{D}]^{1/6}

Where, κ-squared is the relative orientation between the transition dipoles of the donor and acceptor, J(λ) is the overlap integral in the region of the donor emission and acceptor absorbance spectra (with the wavelength expressed in nanometers), η represents the refractive index of the medium, and Q_{D} is the donor quantum yield. Summarizing the basic concepts of RET one can critically relate the rate of energy transfer with the important parameters (κ², J(λ), η, and Q_{D}), of which the Q_{D} dependence is taken advantage of in the current work. Because of the sixth-root dependence in the calculation of R₀, small errors or uncertainties in the value of Q_{D} do not have a large effect on the overall BRET efficiency. Our results clearly indicate that, as a result of utilizing a variant of the donor protein with a 35-fold gain in donor quantum yield, marked improvement in the overall efficiency of the BRET system results, at least when the acceptor moiety is a variant of Aequorea GFP. Clz400 has been previously known to result in extraordinary low light output when used with native RLUC, which stems mainly from poor quantum yield with this substrate (Table 1, FIG. 18).

Previous work in our laboratory employing a strategy of consensus sequence based semi-rational mutagenesis of Renilla luciferase resulted in identification of mutations that greatly increased the quantum yield of Renilla luciferase (Protein Eng Des Sel 2006; 19:391-400, incorporated herein by reference for the corresponding discussion), especially when used with Clz400. During this study we picked these previously identified variants of Renilla luciferase as BRET donors to verify the Q_{D} dependence of the BRET signal, while obtaining a photon efficient BRET vector. Among the vectors generated, as the RLUC8 protein is ˜1 order of magnitude more stable than RLUC in the cytoplasmic environment, use of the double mutation RLUC (C124A/M185V) as a BRET pair with GFP² could be useful for applications where stability of the donor protein is not preferred. The BRET vector using RLUC8, which exhibits increased stability and a 60-fold improvement in light output with the Clz400 substrate, results in the highest improvement in the BRET signal. Previously, we attempted BRET signal detection from mouse models (FASEB J. 2005; 05-4628fje, incorporated herein by reference for the corresponding discussion) using a GFP²-RLUC vector and observed that both the minimum numbers of detectable cells and the required image acquisition time were relatively high. The current BRET vector has shown significant improvements to overcome each of these limitations, with significantly lower number of cells constitutively over-expressing the BRET partners needed for detectability and/or reduced scan-times. Furthermore, improvements were also observed for imaging the BRET signal from deep tissue structures, where both robust acceptor signal and attenuated emission donor signal was captured from the lungs.

Transfection of mammalian cells with the GFP² fusion plasmids utilizing Rluc-M and Rluc8 as the donor, in comparison to the native Rluc containing fusion plasmid, confirms that significantly higher (25-fold and 35-fold respectively) donor light output is translated into higher acceptor light output. Interestingly, the fold gain in observed acceptor light output from the GFP2-Rluc-M and GFP²-Rluc8 constructs is even higher (40 and 80-fold respectively), resulting in a 3.3 and a 5.5-fold increase in the BRET ratio, respectively. Further, by comparing the light signals from stable HT1080 cells with equivalent expression of the donor and acceptor proteins, each cell that expresses GFP²-RLUC8 shows about a 24-fold higher donor signal and 30-fold higher acceptor signal in comparison to GFP²-RLUC expressing cells. Considering results from stable cells as less error-prone, these results clearly indicate that the increased donor quantum yield does make a significant difference in BRET acceptor signal.

By utilizing the GFP²-Rluc8 vector, we attempted live cell imaging of single cells directly from culture dishes by diluting the HT1080 stable cells to very low densities. The results indicate that both donor and acceptor signals from individual cells are much higher than the background signal and thus can be spectrally resolved. Previously, single cell imaging of bioluminescent light has been attempted using microscopes attached with a CCD, where the resultant luminescence signal comes from a direct donor-acceptor fusion or by transcriptional control of a circadian rhythm gene. With the new BRET vector described in the current work, we were able to demonstrate for the first time that BRET signal as a function of protein conformational changes can also be monitored from single cells using a cooled CCD. As a demonstration model, we choose two mTOR pathway proteins, where the mTOR-targeting molecule rapamycin was demonstrated to work as a gain-of-function mechanism in which it binds to the intracellular protein FKBP12. The FKBP12-rapamycin complex is known to form hetero-dimers with a FRAP binding domain called FRB, an event which is documented here from single intact cells. Evaluation of drug response from single cells can help to study heterogeneous cell behavior in cell culture and leads to improved sensitivity for in vivo applications allowing the study of much fewer cells in living animal models. For most cases, by quantitating the signal intensities, it is possible to differentiate the numbers of cells residing at each location. However, as the minimum FOV of the camera is 4 cm diameter, sub-cellular resolution is hard to achieve with the current imaging instrument.

The new BRET vector developed in the current work should be ideal for use as a sensitive assay in vitro, for single live cells in vivo, as well as from living population of cells within small living subjects. The added sensitivity to the known BRET system should also empower drug screening from 384-well plates with few live cells per well, constitutively over-expressing genetic sensors, enabling an automated imaging strategy for high-throughput application of BRET technology.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In addition, the phrase “about ‘x’ to ‘y’” includes “about ‘x’ to about ‘y’”.

Many variations and modifications may be made to the above-described embodiments. All such modifications and variations are intended to be included herein within the scope of this disclosure.

We claim the following:

1. A composition, comprising:
a BDC derivative represented by structure selected from the group consisting of: structure A and structure B: 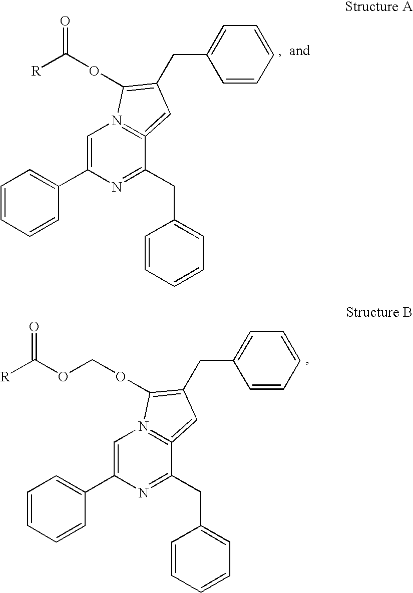
wherein R is selected from the group consisting of:
CH₃, CH₃(CH₂)ₙ, 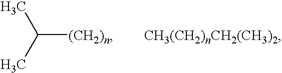
CH₃(CH₂)ₙ(CH═CH)ₙ(CH₂)ₙ, CH₃(CH₂)ₙ(C≡C)ₙ(CH₂)ₙ, 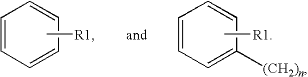
wherein up to five of the carbons on the benzene ring are attached to an independent R1 group, wherein one or more of the R1 groups are each independently selected from the group consisting of: an electron withdrawing group, an electron donating group, and a small alkyl group, and wherein the subscript n is from 1 to 10.

2. The composition of claim 1, wherein the BDC derivative is selected from the group consisting of: acetyl-bisdeoxycoelenterazine, O-Boc-bisdeoxycoelenterazine, acetoxymethyl-bisdeoxycoelenterazine, and pivaloyloxymethyl-bisdeoxycoelenterazine.

3. A BRET system, comprising:
a Renilla luciferase protein, mutant, variant, or derivative thereof,
a fluorescent protein, mutant, variant, or derivative thereof, and
a BDC derivative represented by structure selected from the group consisting of: structure A and structure B: 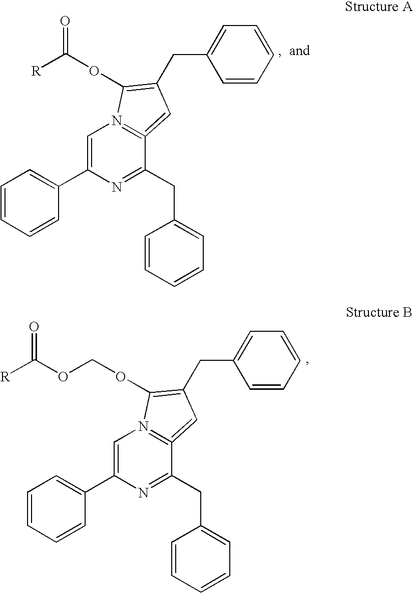
wherein R is selected from the group consisting of:
CH₃, CH₃(CH₂)ₙ, 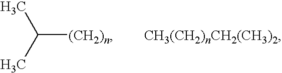
CH₃(CH₂)ₙ(CH═CH)ₙ(CH₂)ₙ, CH₃(CH₂)ₙ(C≡C)ₙ(CH₂)ₙ, 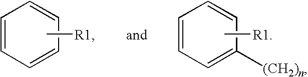
wherein up to five of the carbons on the benzene ring are attached to an independent R1 group, wherein one or more of the R1 groups are each independently selected from the group consisting of: an electron withdrawing group, an electron donating group, and a small alkyl group, and wherein the subscript n is from 1 to 10.

4. The BRET system of claim 3, wherein the fluorescent protein is a green fluorescent protein.

5. The BRET system of claim 3, wherein the BDC derivative is selected from the group consisting of: acetyl-bisdeoxycoelenterazine, O-Boc-bisdeoxycoelenterazine, acetoxymethyl-bisdeoxycoelenterazine, and pivaloyloxymethyl-bisdeoxycoelenterazine.

6. A kit, comprising:
a Renilla luciferase protein, mutant, variant, or derivative thereof,
a fluorescent protein, mutant, variant, or derivative thereof, and
a BDC derivative represented by structure selected from the group consisting of: structure A and structure B: 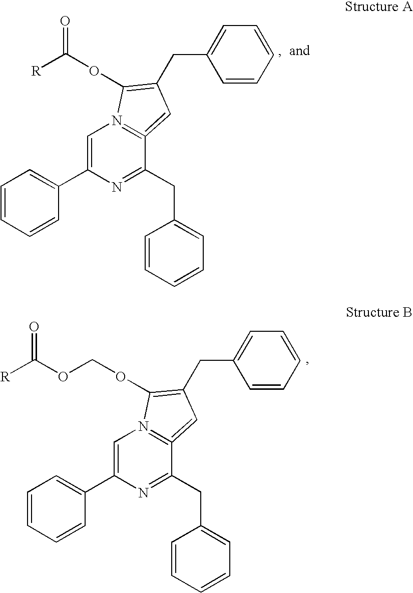
wherein R is selected from the group consisting of:
CH₃, CH₃(CH₂)ₙ, 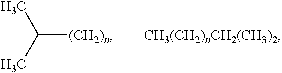
CH₃(CH₂)ₙ(CH═CH)ₙ(CH₂)ₙ, CH₃(CH₂)ₙ(C≡C)ₙ(CH₂)ₙ, 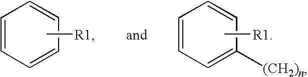
wherein up to five of the carbons on the benzene ring are attached to an independent R1 group, wherein one or more of the R1 groups are each independently selected from the group consisting of: an electron withdrawing group, an electron donating group, and a small alkyl group, and wherein the subscript n is from 1 to 10.

7. The kit of claim 6, wherein the fluorescent protein is a green fluorescent protein.

8. The kit of claim 6, wherein the BDC derivative is selected from the group consisting of: acetyl-bisdeoxycoelenterazine, O-Boc-bisdeoxycoelenterazine, acetoxymethyl-bisdeoxycoelenterazine, and pivaloyloxymethyl-bisdeoxycoelenterazine.
